(19) United States
(12) Patent Application Publication
(10) Pub. No.: US 2015/0343112 A1
(43) Pub. Date: Dec. 3, 2015

(54) LIQUID BANDAGE

(71) Applicant: PROBLEM SOLVING PRODUCTS, LLC, Atlanta, GA (US)

(72) Inventors: Stephen Craig Dyar, Savannah, GA (US); Kiowa KAVOVIT, Studio City, CA (US); Andrew KAVOVIT, Studio City, CA (US); Curtis E. JONES, Savannah, GA (US); John Patrick KENNEDY, Pooler, GA (US)

(73) Assignee: PROBLEM SOLVING PRODUCTS, LLC, Atlanta, GA (US)

(21) Appl. No.: 14/293,423

(22) Filed: Jun. 2, 2014

(51) Int. Cl. A61L 26/00

(52) U.S. Cl. CPC A61L 26/0023; A61L 26/0009; A61L 26/0066; A61L 2300/402

(57) ABSTRACT

According to at least one exemplary embodiment, a composition for a liquid bandage may be described, and may include a composition for a colored liquid bandage. The liquid bandage may be applied to protect an affected area. Additionally, one exemplary embodiment may include a method for making a composition for a liquid bandage. Additionally, one exemplary embodiment may include a method for applying a liquid bandage. Additionally, one exemplary embodiment may include a method for removing a liquid bandage.

### BACKGROUND

Bandages of several varieties are used to protect and heal affected areas, for example: a hangnail; a flesh wound, such as a minor cut or a scrape; or a flesh area, such as an area susceptible to blisters or calluses. Traditional bandage strips are often problematic in that their bulkiness and inflexibility inhibits bandage wearer movement. Further, traditional bandage strips are susceptible to loosening after getting wet.

To address these issues with traditional bandage strips, liquid bandages have been developed. However, currently available liquid bandages lack adequate durability, flexibility, water resistance, and color. Further, current liquid bandages, to varying degrees, sting on application, are slow to dry, and do not conceal the wound.

### SUMMARY

According to at least one exemplary embodiment, a composition for a liquid bandage may be described, and may include a composition for a colored liquid bandage. The liquid bandage may be applied to protect an affected area. Additionally, one exemplary embodiment may include a method for making a composition for a liquid bandage. Additionally, one exemplary embodiment may include a method for applying a liquid bandage. Additionally, one exemplary embodiment may include a method for removing a liquid bandage.

### DETAILED DESCRIPTION OF THE EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the term “embodiments of the invention” does not require that all embodiments of the invention include the discussed feature, advantage or mode of operation.

Exemplary embodiments disclosed herein may provide liquid bandage compositions and methods. Some exemplary embodiments may provide for various compositions themselves. Other exemplary embodiments may provide methods for manufacturing the compositions. Still other exemplary embodiments may provide methods for use of the compositions.

Some exemplary embodiments may be compositions that include one or more alcohols, for example ethyl alcohol. These embodiments may be compositions that include an amount of branched or unbranched aliphatic alcohol that is between about 20 percent by weight (“wt %”) and about 63 wt %, for example compositions that include between about 25 wt/o and about 58 wt % ethyl alcohol, or about 27 wt % and about 48 wt % ethyl alcohol.

Some further exemplary embodiments may be compositions that include one or more alkanes, for example heptane. These embodiments may be compositions that include an amount of heptane that is between 0 wt % and about half of the ethyl alcohol wt % contained therein, for example compositions that include about a 2:1 ratio of ethyl alcohol wt % to heptane wt %. In a further example, compositions that include between about 49 wt % and about 58 wt % ethyl alcohol can also include between about 24 wt % and about 29% heptane. The alkane(s) and/or alcohol(s) used may act as solvents for the composition, or as desired.

Some further exemplary embodiments may be compositions that include one or more chlorinated hydrocarbons, for example methylene chloride. Methylene chloride, when utilized in the exemplary embodiments, can provide superior performance, including, but not limited to, less pain, superior film formation and superior barrier properties, which can be critical to bandages. These embodiments may be compositions that include an amount of methylene chloride that is between 1 to 5 times the wt % of the ethyl alcohol contained therein, for example compositions that include about a 1:2 ratio of ethyl alcohol wt % to methylene chloride wt %. In a further example, compositions that include between about 27 wt % and about 45 wt % ethyl alcohol can also include between about 39 wt % and about 46% methylene chloride. Unexpectedly, it was found that pain (wound stinging sensation) was significantly reduced by maintaining the concentration of methylene chloride equivalent or higher than the branched or unbranched aliphatic alcohol(s) including ethanol, propanol, and isopropyl alcohol. Further, keeping the concentration of methylene chloride equal or greater than any organic solvent(s) known to sting, produced similar significant reduction in stinging pain when the combinations were employed. More surprisingly, the pain reduction was greater than using methylene chloride alone. Such higher concentrated methylene chloride ratios also provided unexpected reductions in drying time. Neither pain reduction nor improved drying times were attributable to simple chemical phenomena such as azeotropic behavior.

Some exemplary embodiments may also be compositions that include at least one film former, for example a polymer or resin, for example shellac or cellulose acetate. Cellulose acetate, when utilized in at least one exemplary embodiment, can provide superior performance, including, but not limited to, superior film formation and enhanced robustness to abrasion which can be critical to bandages. Embodiments may be compositions that include an amount of film former that is between about 5 wt % and about 40 wt %, for example compositions that include an amount shellac or cellulose acetate that is between about 5 wt % and about 40 wt %, and further, for example compositions that include an amount of shellac or cellulose acetate that is between about 10 wt % and about 25 wt %.

Some further exemplary embodiments may be compositions that include clove oil and/or eugenol. Known for anesthetic qualities, when utilized in the exemplary embodiments, these components can have great value as co-solvents that ultimately provide for superior film formation and superior barrier properties which are critical to bandages. These embodiments may be compositions that include an amount of clove oil and/or eugenol that is between 0.25 wt % and as high as about 70 wt %. Further, some exemplary embodiments may be compositions that include a colorant or pigment. Embodiments may be compositions that include an amount of colorant or pigment that is between 0 wt % and about 10 wt %, for example compositions that include an amount of colorant or pigment that is between about 3 wt % and about 7 wt %.

Also, some exemplary embodiments may be compositions that include one or more antimicrobial agent, for example alcohol, benzalkonium chloride, benzethonium chloride, camphorated metacresol, camphorated phenol, eucalyptol, hexylresorcinol, hydrogen peroxide, iodine, an iodophor, isopropyl alcohol, menthol, methylbenzethonium chloride, methyl salicylate, phenol, and thymol. Some exemplary compositions may include an antimicrobial that is between 0.1 wt % and as high as 70 wt %, or as desired.

Also, some exemplary embodiments may be compositions that include one or more plasticizers, for example castor oil, triethylcitrate or the like. Further, some exemplary embodiments may be compositions that include one or more opacifiers, for example fumed silica or the like. Still further, some exemplary embodiments may be compositions that include one or more thickeners, for example hydroxypropylcellulose, calcium acetate, fumed silica or the like.

Some exemplary embodiments of may be the compositions described in the following tables. The values shown in these tables may be appreciated to be exemplary and variation in the amounts and ratios are contemplated in further exemplary embodiments. All of the following examples may have the advantages of low odor, less pain, stability, and ease of application. The methylene chloride formulations may also be non-flammable, a considerable advantage for pediatric populations. Unexpectedly, it was found that shellac formulations may have the distinct advantage of being able to accept additional coats even hours after the original application to touch up or repair a worn bandage. It was found that the shellac formulations may have a self-healing attribute upon reapplication that far exceeds other film formers. The repairs may be indistinguishable from the original application as the newly applied material coalesces into the base coat of the original bandage. Alternatively, reapplication of the shellac formulations may also be used to remove the original bandage if wiped clean immediately after application, another unique quality to the system.

TABLE 1

| Ingredient | Percent (%) | weight (gm) |
|---|---|---|
| Heptane | 27% | 1.750 |
| Ethyl Alcohol | 53% | 3.500 |
| Shellac | 15% | 1.000 |
| Color | 5% | 0.300 |
| Total | 100% | 6.550 |

TABLE 2

| Ingredient | Percent (%) | weight (gm) |
|---|---|---|
| Heptane | 32% | 1.750 |
| Ethyl Alcohol | 63% | 3.500 |
| Shellac | 5% | 0.300 |
| Color | 0% | 0.000 |
| Total | 100% | 5.550 |

TABLE 3

| Ingredient | Percent (%) | weight (gm) |
|---|---|---|
| Heptane | 18% | 1.750 |
| Ethyl Alcohol | 36% | 3.500 |
| Shellac | 36% | 3.500 |
| Color | 10% | 1.000 |
| Total | 100% | 9.750 |

TABLE 4

| Ingredient | Percent (%) | weight (gm) |
|---|---|---|
| Heptane | 29% | 1.750 |
| Ethyl Alcohol | 58% | 3.500 |
| Shellac | 10% | 0.600 |
| Color | 3% | 0.200 |
| Total | 100% | 6.050 |

TABLE 5

| Ingredient | Percent (%) | weight (gm) |
|---|---|---|
| Heptane | 24% | 1.750 |
| Ethyl Alcohol | 49% | 3.500 |
| Shellac | 20% | 1.400 |
| Color | 7% | 0.500 |
| Total | 100% | 7.150 |

TABLE 6

| Ingredient | Percent (%) | weight (gm) |
|---|---|---|
| Methylene Chloride | 42% | 3.500 |
| Ethyl Alcohol | 42% | 3.500 |
| Shellac | 12% | 1.000 |
| Color | 4% | 0.300 |
| Total | 100% | 8.300 |

TABLE 7

| Ingredient | Percent (%) | weight (gm) |
|---|---|---|
| Methylene Chloride | 47% | 3.500 |
| Ethyl Alcohol | 47% | 3.500 |
| Shellac | 4% | 0.300 |
| Color | 0% | 0.000 |
| Total | 100% | 7.300 |

TABLE 8

| Ingredient | Percent (%) | weight (gm) |
|---|---|---|
| Methylene Chloride | 30% | 3.500 |
| Ethyl Alcohol | 30% | 3.500 |
| Shellac | 30% | 3.500 |
| Color | 9% | 1.000 |
| Total | 100% | 11.500 |

TABLE 9

| Ingredient | Percent (%) | weight (gm) |
|---|---|---|
| Methylene Chloride | 45% | 3.500 |
| Ethyl Alcohol | 45% | 3.500 |
| Shellac | 8% | 0.600 |
| Color | 3% | 0.200 |
| Total | 100% | 7.800 |

TABLE 10

| Ingredient | Percent (%) | weight (gm) |
|---|---|---|
| Methylene Chloride | 39% | 3.500 |
| Ethyl Alcohol | 39% | 3.500 |
| Shellac | 16% | 1.400 |
| Color | 6% | 0.500 |
| Total | 100% | 8.900 |

TABLE 11

| Ingredient | Percent (%) | weight (gm) |
|---|---|---|
| Methylene Chloride | 42% | 3.500 |
| Ethyl Alcohol | 42% | 3.500 |
| Cellulose Acetate | 12% | 1.000 |
| Color | 4% | 0.300 |
| Total | 100% | 8.300 |

TABLE 12

| Ingredient | Percent (%) | weight (gm) |
|---|---|---|
| Methylene Chloride | 39% | 3.500 |
| Ethyl Alcohol | 39% | 3.500 |
| Cellulose Acetate | 11% | 1.000 |
| Eugenol | 7% | 0.600 |
| Color | 3% | 0.300 |
| Total | 100% | 8.900 |

TABLE 13

| Ingredient | Percent (%) | weight (gm) |
|---|---|---|
| Methylene Chloride | 39% | 3.500 |
| Ethyl Alcohol | 39% | 3.500 |
| Shellac | 11% | 1.000 |
| Eugenol | 7% | 0.600 |
| Color | 3% | 0.300 |
| Total | 100% | 8.900 |

TABLE 14

| Ingredient | Percent (%) | weight (gm) |
|---|---|---|
| Methylene Chloride | 38% | 3.500 |
| Ethyl Alcohol | 38% | 3.500 |
| Cellulose Acetate | 11% | 1.000 |
| Shellac | 11% | 1.000 |
| Color | 3% | 0.300 |
| Total | 100% | 9.300 |

TABLE 15

| Ingredient | Percent (%) | weight (gm) |
|---|---|---|
| Methylene Chloride | 41% | 3.500 |
| Ethyl Alcohol | 41% | 3.500 |
| Fumed Silica | 3% | 0.300 |
| Shellac | 12% | 1.000 |
| Color | 3% | 0.300 |
| Total | 100% | 8.600 |

TABLE 16

| Ingredient | Percent (%) | weight (gm) |
|---|---|---|
| Methylene Chloride | 49% | 490.0 |
| Ethyl Alcohol | 29% | 290.0 |
| Triacetin | 2% | 20.0 |
| Shellac | 19.5% | 195.0 |
| TLB-170000 (pigment) | 0.5% | 5.0 |
| Total | 100% | 1000.0 |

Any exemplary embodiment disclosed herein may be a composition that may be stored at room temperature, away from fire or open flames, and away from heat.

Some embodiments may include a method for applying the liquid bandage disclosed herein. One exemplary embodiment of a method for applying the liquid bandage may include first applying a stencil, exposing the wound inside a shape within the stencil, then applying the liquid bandage and removing the stencil to leave the bandage in a silhouetted shape. The stencil may further be fabricated from flexible film and may be coated on one size with a gentle adhesive to fix the film in place while applying the liquid bandage in the void space of the stencil. Such methods are may be especially useful for pediatric patients to make the application of a liquid bandage of lesser anxiety by removing the focus from the wound itself. Stencils of lady bugs, lightening bolts, and animals are non-limiting examples.

Some additional embodiments may include a method for making compositions disclosed herein. One exemplary embodiment of a method for making the compositions may include weighing an amount of ethyl alcohol to a desired mass and placing a weighed amount of ethyl alcohol in a mixing container. Further, this embodiment may include weighing an amount of film former to a desired mass and placing a weighed amount of film former in the mixing container. This embodiment may include a film former from at least one polymer or resin, for example shellac and cellulose acetate.

Still further, this exemplary embodiment may include mixing the weighed amounts of ethyl alcohol and film former in the mixing container using a combination of a vortex mixer and sonication until the weighed amount of film former dissolves in the weighed amount of ethyl alcohol to form a solution. Also, this embodiment may include heating of the weighed amounts of ethyl alcohol and shellac in the mixing container up to about 78° C., which in some embodiments can be critical insofar as speeding up the dissolution of the shellac, for example to a commercially viable time.

Additionally, this embodiment may include weighing an amount of heptane or methylene chloride to a desired mass and adding a weighed amount of heptane to the mixing container with the alcohol solution. Further, this embodiment may include mixing the weighed amount of heptane or methylene chloride with the alcohol solution in the mixing container using a vortex mixer until all contents are well-mixed to form an intermediate formulation.

Moreover, this embodiment may include weighing an amount of colorant or pigment to a desired mass and adding a weighed amount of colorant or pigment to the mixing container with the intermediate formulation. Further, this embodiment may include mixing the weighed amount of colorant or pigment with the intermediate formulation in the mixing container using a vortex mixer and sonication to form a liquid bandage formulation. Still further, this embodiment may include transferring the liquid bandage formulation to a dispensing container.

Some exemplary embodiments may be a method for using compositions disclosed herein. One embodiment of a method for using a composition may include cleaning an affected area, for example: a hangnail; a flesh wound, such a paper cut, a minor cut or a scrape; or a flesh area, such as an area susceptible to blisters or calluses. Further, this embodiment may include shaking a dispensing container that contains a composition disclosed herein. Additionally, this embodiment may include applying an initial layer of a composition contained in the dispensing container on the affected area and then allowing the initial layer to dry to touch. Still further, this embodiment may include applying an additional layer of a composition on the initial layer after the initial layer has dried to touch. Likewise, this embodiment may include applying multiple additional layers of a composition on top of a previously applied and dried layer of a composition. This embodiment may be a colored liquid bandage, which may include one or more applied and dried layers of a composition disclosed herein.

Another exemplary embodiment of a method for using a composition may be removing an existing colored liquid bandage. This embodiment may include applying a new layer of a composition on the existing colored liquid bandage and then, immediately thereafter, wiping the new layer and the existing colored liquid bandage away. Further, this embodiment may include applying a layer of suitable solvent, for example nail polish remover, on the existing colored liquid bandage and then, immediately thereafter, wiping layer of nail polish remover and the existing colored liquid bandage away. Also, this embodiment may include applying a layer of nail polish remover and a composition on the existing colored liquid bandage and then, immediately thereafter, wiping layer of nail polish remover, a composition, and the existing colored liquid bandage away.

The foregoing description illustrates the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A composition for a liquid bandage comprising:
at least one film former;
at least one non-aqueous solvent; and
at least one pigment.

2. The composition of claim 1, wherein the at least one film former comprises at least one of shellac or cellulose acetate.

3. The composition of claim 1, wherein the at least one film former is between about 5 wt % and 40 wt % of the composition.

4. The composition of claim 1, wherein the at least one non-aqueous solvent comprises at least one of:
an alcohol;
an alkane; or
a chlorinated hydrocarbon.

5. The composition of claim 4, wherein the at least one non-aqueous solvent comprises a branched or unbranched aliphatic alcohol.

6. The composition of claim 5, wherein the at least one non-aqueous solvent comprises ethyl alcohol.

7. The composition of claim 6, wherein the ethyl alcohol is between about 20 wt % and 63 wt % of the composition.

8. The composition of claim 4, wherein the at least one non-aqueous solvent comprises heptane.

9. The composition of claim 4, wherein the at least one non-aqueous solvent comprises methylene chloride.

10. The composition of claim 9, wherein methylene chloride comprises at least 50% of the total solvent.

11. The composition of claim 1, further comprising an antimicrobial agent.

12. A composition for a liquid bandage comprising:
at least one film former;
a first non-aqueous solvent; and
a second non-aqueous solvent.

13. The composition of claim 12, wherein the first non-aqueous solvent comprises ethyl alcohol and the second non-aqueous solvent comprises heptane.

14. The composition of claim 12, wherein the first non-aqueous solvent comprises methylene chloride.

15. The composition of claim 14, wherein the second non-aqueous solvent comprises a branched or unbranched aliphatic alcohol.

16. The composition of claim 12, wherein at least one of the first non-aqueous solvent and the second non-aqueous solvent is one of:
clove oil; or
eugenol.

17. The composition of claim 12, further comprising a pigment.

18. The composition of claim 12, further comprising an antimicrobial agent.

19. A method for applying a liquid bandage, comprising:
applying a stencil over a desired area;
applying a layer of the liquid bandage to the exposed area within the void space of the stencil; and
removing the stencil;
wherein following the removal of the stencil, the layer of the liquid bandage on the desired area has a distinct silhouette shape.

20. The method of claim 19, further comprising applying a second layer of the liquid bandage to the exposed area within the void space of the stencil.
